(19) United States
(12) Patent Application Publication
(10) Pub. No.: US 2022/0371026 A1
(43) Pub. Date: Nov. 24, 2022

(54) APPARATUSES SYSTEMS AND METHODS USING CORE-SHELL-SHELL MAGNETIC BEADS

(71) Applicant: Apostle, Inc., Menlo Park, CA (US)

(72) Inventors: Dongliang Ge, Redwood City, CA (US); Hao Wan, Palo Alto, CA (US); Shuting Zhao, Santa Clara, CA (US); Bo Zhang, San Carlos, CA (US); Xin Guo, Palo Alto, CA (US)

(73) Assignee: Apostle, Inc., Menlo Park, CA (US)

(21) Appl. No.: 17/761,381

(22) PCT Filed: Jul. 10, 2020
(86) PCT No.: PCT/US2020/041682

§ 371 Date: Mar. 17, 2022

Related U.S. Application Data
(60) Provisional application No. 62/902,136, filed on Sep. 18, 2019.
(60) Provisional application No. 62/932,218, filed on Nov. 7, 2019.

(51) Int. Cl. B03C 1/01; C07K 17/08; C12N 15/10

(52) U.S. Cl. CPC B03C 1/01; C07K 17/08; C12N 15/1013

(57) ABSTRACT

In some examples, a CSS-MBs includes a solid magnetic core, a first shell material which surrounds the solid magnetic core and a second shell material which surrounds the first shell material. The first shell material may be a protective layer. The first shell material may include an inert carbon material. The second shell material may be have surface chemistry which allows for selective interaction of the CSS-MB with certain biomolecules under various buffer conditions.

### CROSS-REFERENCE TO RELATED APPLICATION

This application claims the filing benefit of U.S. Provisional Application No. 62/902,136, filed Sep. 18, 2019 and U.S. Provisional Application No. 62/932,218, filed Nov. 7, 2019. These applications are incorporated by reference herein in their entireties and for all purposes.

### BACKGROUND

There may be many applications where it is useful to separate target biomolecules from a solution or to enrich the population of target biomolecules within a solution. Many biological solutions (e.g., patient samples, laboratory mixtures, etc.) may include a mix of target biomolecules along with other non-target components (non-targeted biomolecules, salts, buffer components, cells, etc.). Different procedures have been developed with the goal of extracting and/or enriching the target biomolecules, for example to remove undesirable components of the original solution and/or to increase the proportion of the target products compared to non-target products.

Various types of bead may be useful for the separation (and/or enrichment) of biomolecules. For example, the beads may bind to the biomolecules since it may be easier to separate the bound beads from the solution than it would be to separate the biomolecules alone. The bound beads may respond to a force which the biomolecule alone does not respond to. The characteristics of the beads, such as the biomolecules they bind and the forces they respond to, may be dictated, in part, by the composition and chemistry of the beads.

### SUMMARY

In at least one aspect, the present disclosure relates to a magnetic bead including a solid magnetic core, a first shell surrounding the magnetic core, and a second shell surrounding the first shell.

The first shell may include a protective layer. The first shell may be non-porous. The solid magnetic core may include a metal alloy oxide with the composition Feₓ—Co_{y}—NiO_{z}. The first shell may include an inert carbon shell. The second shell may include a silica oxide shell.

The magnetic bead may have a diameter of 1 μm or less. The solid magnetic core has a diameter of 100-300 nm, the first shell has a thickness of 10-50 nm, and the second shell has a thickness of 20-100 nm. The first shell may include hydroxyl groups, epoxy groups, or combinations thereof. The second shell may include mesopores. The mesopores may be between 2-20 nm.

The magnetic beads may include chemical groups disposed on an outer surface of the second shell. The chemical groups may include hydrophilic moieties selected from the group comprising polyethylene glycol, polyacrylic acid, polyvinyl alcohol, glucose, sucrose, cysteine, maltose, chitosan, alginate, cellulose, chitin, starch and combinations thereof. The chemical groups may include compounds with epitope carboxylic groups selected from the group comprising succinic anhydride, polyacrylic acid, amino acids, alginic acid, carbonic acid, malic acid, tartaric acid, citric acid, salicylic acid, gallic acid, sialic acid.

In at least one aspect, the present disclosure relates to a method which includes forming a magnetic core, coating the magnetic core with a first shell material, and coating the first shell material with a second shell material.

Forming the magnetic core may include heating a mixture of iron salts and salts of at least one other metal under pressure. The mixture of iron salts may include Iron (III) Chloride, Iron (III) Sulfate, Iron (III) Nitrate and combinations thereof. The mixture of iron salts and salts of at least one other metal may include a surfactant.

The method may also include purifying the magnetic core. Coating the magnetic core with the first shell material may include a solvothermal carbonization of a sugar onto the magnetic core. The sugar may be glucose, sucrose, maltose, or combinations thereof.

The method may also include coating a polymer material onto the magnetic core. Coating the magnetic core with the first shell material may include coating the magnetic core and polymer material with the first shell material. Coating the first shell material with the second shell material may include decomposing the second shell material in a solution onto an intermediate bead including the magnetic core and first shell material. The second shell material may include a silicon organic compound selected from the group comprising tetraethyl orthosilicate, tetramethyl orthosilicate, and combinations thereof.

The method may also include forming pores in the second shell material. The method may also include modifying a surface of the second shell material. The surface modification may include converting hydroxyl groups on a surface of the second shell material to active sites and binding functional groups to the active sites.

In at least one aspect, the present disclosure may relate to a method. The method includes mixing core-shell-shell magnetic beads (CSS-MBs) with a sample including target biomolecules. The CSS-MBs include a solid magnetic core, a first shell surrounding the solid magnetic core, and a second shell surrounding the first shell. The method includes applying a magnetic field to the mixture to concentrate the CSS-MBs into a pellet, separating the pellet from a supernatant of the mixture, and recovering the target biomolecules from the pellet.

The target biomolecule may include nucleic acids, proteins, peptides, cells, exosomes, or combinations thereof. The method may include binding biomolecules above a cutoff size to the CSS-MBs in the presence of a binding buffer. Recovering the target biomolecules may include eluting the target biomolecules from the CSS-MBs in the presence of an elution buffer. The target biomolecules may be nucleic acids, and the binding buffer may include a nucleic acid precipitation buffer including a dehydrating agent, a salt bridge, a buffering agent, carrier molecules, a surfactant, and combinations thereof. The method may include washing the pellet with a wash buffer.

In at least one aspect, the present disclosure may relate to a kit including a population of core-shell-shell magnetic beads (CSS-MBs) and a nucleic acid precipitation reagent. Each CSS-MB includes a solid magnetic core, a first shell material surrounding the solid magnetic core, and a second shell material surrounding the first shell material. The CSS-MBs may reversibly bind nucleic acids in the presence of the nucleic acid precipitation reagent.

The nucleic acid precipitation reagent may include a dehydrating agent, a salt bridge, a buffering agent, carrier molecules, a surfactant, and combinations thereof. The kit may also include a wash buffer. The kit may also include an elution buffer. The CSS-MBs may release the bound nucleic acids in the presence of the elution buffer.

### BRIEF DESCRIPTION OF THE DRAWINGS

FIG. 1 is a cross-sectional diagram of a core-shell-shell magnetic bead according to some embodiments of the present disclosure.

FIG. 2 is a flow chart of a method of synthesizing core-shell-shell magnetic beads according to some embodiments of the present disclosure.

FIGS. 3A and 3B are electron micrographs of core-shell-shell magnetic beads according to some embodiments of the present disclosure.

FIG. 4 is a spectrograph of a core-shell-shell magnetic bead according to some embodiments of the present disclosure.

FIG. 5 is a graph of the size distribution of core-shell-shell magnetic beads according to some embodiments of the present disclosure.

FIG. 6 is a flow chart depicting a method of enriching biomolecules according to some embodiments of the present disclosure.

FIG. 7 is a graph of core-shell-shell magnetic beads used to separate nucleic acids by size according to some embodiments of the present disclosure.

FIG. 8 is a graph of using core-shell-shell magnetic beads to recover nucleic acids between different cutoff ranges according to some embodiments of the present disclosure.

FIG. 9 is a graph of a nucleic acids before and after clean up with core-shell-shell magnetic beads according to some embodiments of the present disclosure.

### DETAILED DESCRIPTION

The following description of certain embodiments is merely exemplary in nature and is in no way intended to limit the scope of the disclosure or its applications or uses. In the following detailed description of embodiments of the present systems and methods, reference is made to the accompanying drawings which form a part hereof, and which are shown by way of illustration specific embodiments in which the described systems and methods may be practiced. These embodiments are described in sufficient detail to enable those skilled in the art to practice presently disclosed systems and methods, and it is to be understood that other embodiments may be utilized and that structural and logical changes may be made without departing from the spirit and scope of the disclosure. Moreover, for the purpose of clarity, detailed descriptions of certain features will not be discussed when they would be apparent to those with skill in the art so as not to obscure the description of embodiments of the disclosure. The following detailed description is therefore not to be taken in a limiting sense, and the scope of the disclosure is defined only by the appended claims.

Magnetic beads may be useful for the separation and/or enrichment of a target biomolecule. Magnetic beads have a relatively strong magnetic response which allows them to be attracted to (or repelled by) a magnetic field. For example, a magnetic field may be applied to a solution containing magnetic beads, which may cause the magnetic beads to be collected in a region as close as possible to the magnetic field. When a magnetic field is not present, the magnetic beads may be dispersed in the solution. Surface chemistry of the magnetic beads along with other factors (e.g., buffer conditions) may cause certain biomolecules to preferentially bind to the surface of the magnetic beads, which may be exploited to carry out various assays. The binding of the biomolecules to the magnetic beads may be reversible, and the bound biomolecules may later be eluted from the magnetic beads.

Some magnetic beads may generally be composed of polymer (i.e. polystyrene) beads with interspersed multiple small magnetic nanoparticles (20-30 nm). Due to the small size of the individual magnetic nanoparticles, the magnetic response of each magnetic nanoparticle is not very strong. Accordingly, the overall magnetic bead including the magnetic nanoparticles may generally be large (usually around 1-5 μm) to provide enough space to include numerous magnetic nanoparticles to achieve a desired magnetic response. This relatively large size of the magnetic bead results in large sediment rate and low surface area to volume ratio, which limits the efficient interaction of magnetic beads with surrounding biomolecules and decreases the number of binding sites for biomolecules. This may reduce the utility of such beads in various assays where low sedimentation and high binding affinity are required. Furthermore, the use of a polymer material such as polystyrene in the magnetic bead means that functional groups of benzene are exposed on the surface of magnetic beads. The hydrophobic property of benzene would cause strong non-specific binding of biomolecules. For example, this makes nucleic acids with larger size difficult to be released from the magnetic beads after binding and also would potentially sacrifice the binding sites for nucleic acids since they may be ‘filled’ by non-specific binding of other biomolecules (e.g. protein, peptide). There may thus be a need for relatively small magnetic nanoparticles with a high magnetic response and advantageous surface binding properties.

The present disclosure is drawn to apparatuses, systems, and methods for core-shell-shell magnetic beads (CSS-MBs). A CSS-MB may include a solid magnetic core which is surrounded by a first shell, which in turn is surrounded by a second shell. In some embodiments, the solid magnetic core may be a single material, such as a single superparamagnetic crystal. The first shell may be a protective layer which protects the magnetic core from interaction with an external solution (and vice versa). The second shell may provide chemistry for interaction with the target biomolecules. The solid magnetic core may allow relatively small (e.g., about 1 μm or smaller) CSS-MB to exhibit a relatively strong magnetic response. The reduced size of the CSS-MB may decrease the sedimentation rate of the magnetic bead and also increase the surface area to volume ratio of the magnetic bead. The first shell may allow the magnetic bead to be encapsulated, which may allow for more freedom in the chemistry chosen for the second shell. The second shell may allow for improved surface chemistry, which may allow for increased specificity of binding to the target biomolecules. The use of the two shells may allow for beads of relatively small size, which still have a strong magnetic response (due to the solid magnetic core) but which will also have advantageous surface chemistry (due to the separation between the outer shell and the core provided by the first shell). The properties of such a CSS-MB may be advantageous in a number of potential assays.

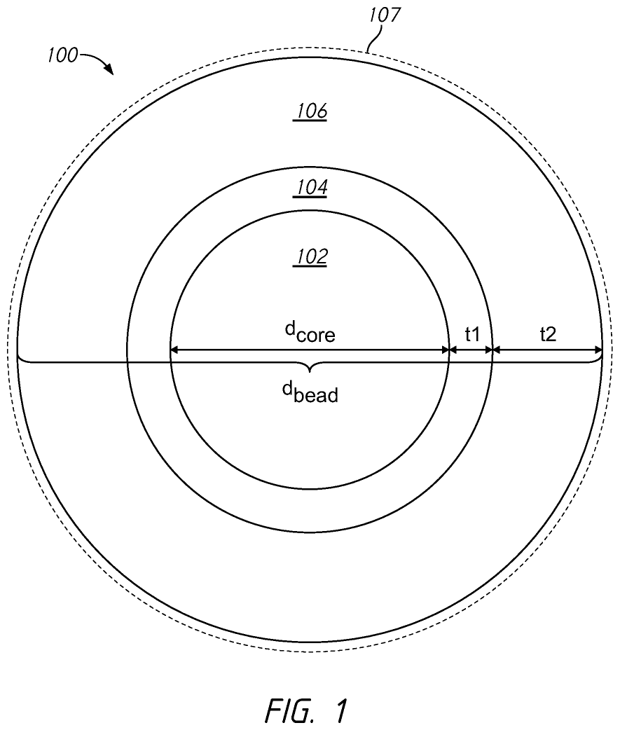

FIG. 1 is a cross-sectional diagram of a core-shell-shell magnetic bead according to some embodiments of the present disclosure. The core-shell-shell magnetic bead (CSS-MB) 100 includes a magnetic core 102, a first shell 104 and an outer shell 106.

In some embodiments, the CSS-MB 100 may have a generally spherical shape, with a size defined by a diameter d_{bead}. The CSS-MBs 100 may have a diameter d_{bead} which is less than about 1 μm. For example, the diameter d_{bead} of a CSS-MB 100 may be between about 200 nm and 1000 nm. Larger and smaller diameters may be used in other example embodiments. It should be understood that while CSS-MBs 100 may generally be shown as spherical for ease of discussion, this may represent an idealized view of a CSS-MB 100, and the CSS-MBs 100 may deviate from perfectly spherical. For example, a population of CSS-MBs 100 may include individual CSS-MBs 100 which are spherical, oblate, prolate, ellipsoid, have one or more other deviations from perfectly spherical (e.g., concave or convex ‘bumps’ on the surface), or combinations thereof.

The magnetic core 102 may have a diameter de of between about 100 nm and 300 nm. The magnetic core 102 may be a solid material. In some embodiments the magnetic core 102 may be a solid cluster of crystals such as a cluster of superparamagnetic crystals. In some embodiments, the magnetic core 102 may be a single superparamagnetic crystal. In some embodiments the magnetic core may be formed of a metallic alloy such as a metal alloy oxide crystals. In some embodiments, the magnetic core 102 may include a mixture of metals at a selected ratio. For example, the magnetic core 102 may include a mixture of metals such as Fe, Co, and Ni. In some embodiments, the magnetic core 102 may generally have the composition Feₓ—Co_{y}—NiO_{z}, where x, y, and z are all integer values.

The first shell 104 may generally be a spherical layer deposited on an outer surface of the magnetic core 102. The first shell 104 may have a thickness t1, which in some embodiments may be between about 10 nm to about 50 nm. The first shell 104 may be a protective shell which prevents chemical interactions between the components of an environment outside the CSS-MB 100 and the magnetic core 102. The first shell 104 may be non-porous. The first shell 104 may be formed from an inert material. For example, the first shell 104 may include graphitic carbon. In some embodiments, the first shell 104 may have an outer surface which includes one or more chemical groups which promote bonding between the first shell 104 and the second shell 106. For example, if first shell 104 includes graphitic carbon, the surface of the first shell 104 may include relatively abundant hydroxyl and/or epoxy groups which may provide anchoring sites for the second shell 106.

The second shell 106 may generally be a spherical layer deposited on an outer surface of the first shell 104. The second shell 106 may have a thickness t2, which in some embodiments may be between about 20 nm to 100 nm. Accordingly, the CSS-MB 100 may have an overall radius that is the radius of the magnetic core 102 plus the thickness t1 and thickness t2 of the first shell 104 and second shell 106 respectively.

The second shell 106 may be an outer shell of the CSS-MP 100. The second shell 106 may further protect the magnetic core 102 (e.g., in addition to the protection offered by the first shell 104), may provide surface chemistry useful for one or more reactions, and/or provide active sites which may be functionalized with additional chemical groups to provide such surface chemistry. In some embodiments, the second shell 106 may be non-porous. In some embodiments, the second shell 106 may be porous. For example, the second shell 106 may include mesopores which may be evenly distributed throughout the second shell 106. In some embodiments the second shell may be formed from silicon oxide, such as a condensed silicon oxide.

In some embodiments, an outer surface the second shell 106 may be surrounded by a layer of chemical groups 107. These chemical groups 107 may be inherent to the chemistry of the second shell 106 and/or may be due to modification of the surface chemistry of the second shell. For example, the chemical groups 107 may include chemicals which are bound to the outer surface of the second shell 106. The surface chemical groups 107 may be used to improve the chemical properties of the CSS-MB 100 in a solution (e.g., their dispersal in solution) and/or to promote the binding between the CSS-MB 100 and one or more biomolecules.

In some embodiments, various functional groups may be bound to the surface of the CSS-MB as part of the chemical groups 107. The second shell 106 may include and/or may be modified to include a number of active sites which may be used to bind the functional groups to the outer surface of the second shell 106. For example, in embodiments where the second shell 106 is formed of silicon dioxide, the second shell 106 may include a number of surface hydroxyl groups. These surface hydroxyl groups may be modified into carboxylic groups, which may form active sites for the binding of one or more functional groups.

One example of chemical groups 107 may include chemicals to promote binding between biomolecules. For example, the surface hydroxyl groups of the CSS-MB 100 may be modified with chemicals presenting carboxylic groups such as small molecules, polymers, dendrimers containing an epitope of carboxylic groups, by covalently binding these chemicals to the hydroxyl groups. For example, the hydroxyl groups may be converted to carboxylic groups with chemicals such as succinic anhydride, polyacrylic acid, amino acids, alginic acid, carbonic acid, malic acid, tartaric acid, citric acid, salicylic acid, gallic acid, sialic acid, and combinations thereof. The carboxylic groups may be useful to enable binding of certain target biomolecules based on the mechanism of solid-phase reverse immobilization under certain buffer conditions.

The chemical groups 107 may also include hydrophilic components (e.g., hydrophilic moieties) which may alter the overall surface chemistry of the CSS-MB 100. For example, the hydrophilic components may be small molecules (e.g. zwitterionic molecule, sugar molecule) or polymers (e.g. polyethylene glycol, poly vinyl alcohol, polyethyleneimine). The hydrophilic components may increase the dispersibility of magnetic beads in aqueous solutions, which may be beneficial for interaction between targeted biomolecules and the CSS-MBs 100. The hydrophilic components in the chemical groups 107 may also help avoid the non-specific binding of other biomolecules in the samples, which in turn may enhance the purity of enriched targeted biomolecules. Example of hydrophilic components include polyethylene glycol, polyacrylic acid, polyvinyl alcohol, glucose, sucrose, cysteine, maltose, chitosan, alginate, cellulose, chitin, starch and combinations thereof.

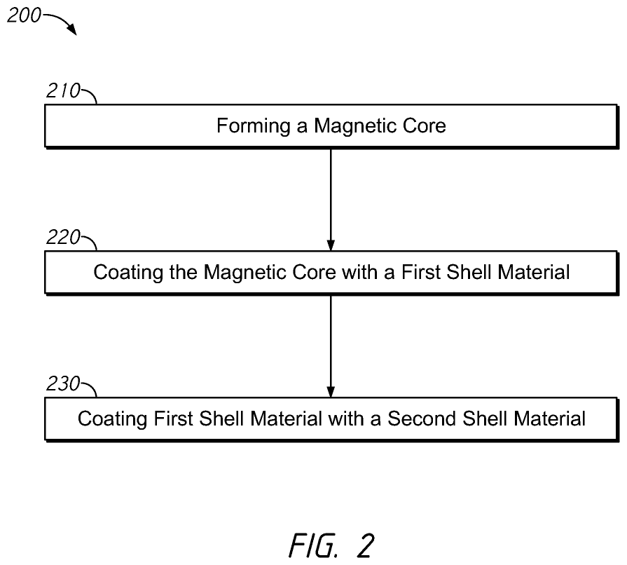

FIG. 2 is a flow chart of a method of synthesizing core-shell-shell magnetic beads according to some embodiments of the present disclosure. The method 200 may, in some embodiments, be used to synthesize core-shell-shell magnetic beads (CSS-MBs) such as the CSS-MBs 100 of FIG. 1.

The method 200 may generally begin with box 210, which describes forming a magnetic core (e.g., magnetic core 102 of FIG. 1). In some embodiments, box 210 may involve a solvothermal synthesis of the magnetic core. The synthesis of the magnetic core described in box 210 may involve a starting mixture of one or more iron salts (e.g., Iron (III) Chloride, Iron (II) Sulfate, Iron (III) Nitrate) and salts of one or more other metals (i.e. Co, Ni) dispersed in an organic solvent (e.g., Ethylene Glycol), in the presence of an ionic capping reagent (i.e. acetate salt). In some embodiments, this mixture may also include a surfactant. In some embodiments, the mixture of metal salts may be obtained as pre-mixed starting material. In some embodiments, the method 200 may include combining one or more reagents to form the mixture of iron salts.

The synthesis described in box 210 may involve heating the mixture of metal salts under pressure to form the magnetic cores. For example, the mixture of metal salts may be transferred into an autoclave reactor, and may be heated under pressure to a temperature between about 180-240° C., for between about 4-80 hours. For example, if the mixture of metal salts includes a mixture of Fe, Co, and Ni salts, a number of metal cores may be formed which include crystals with the general formula Feₓ—Co_{y}—NiO_{z} (where x, y, and z are all integers). The synthesis described in box 210 may include cooling the mixture including the magnetic cores to room temperature. The synthesis described in box 210 may include purifying the magnetic cores, for example by washing the magnetic cores with a wash buffer (e.g., water and/or ethanol), and then drying the magnetic cores. For example, the magnetic cores may be washed by applying a magnetic field to pull the magnetic cores out of solution, removing the supernatant, and suspending the magnetic cores in a wash buffer (e.g., water and/or ethanol). The washing may be repeated multiple times. The dried magnetic cores may yield a solid phase collection of the magnetic cores.

Box 210 may generally be followed by box 220, which describes coating the magnetic core with a first shell material. The first shell material may form the first shell 104 of FIG. 1 in some embodiments. This may create an intermediate bead (e.g., a magnetic core surrounded by a first shell). Box 220 may include solvothermal carbonization of a sugar (i.e. glucose, sucrose, maltose, etc.) onto the magnetic cores. The magnetic cores may be mixed homogenously with sugar. For example, the dried magnetic cores may be mixed mechanically with sugar. The mixture of magnetic cores and sugar may be heated under pressure to carbonize the sugar. For example, the mixture of magnetic cores and sugar may be transferred into an autoclave reactor, and may be heated under pressure to a temperature of between about 100-180° C., for between about 24-60 hours. During the process of heating the mixture under pressure, a carbon shell may be formed onto the magnetic cores.

In some embodiments, box 220 may include coating a polymer material onto the dried magnetic cores, followed by a heat assisted carbonization process in an inert gas environment in order. This may coat the polymer material with a carbon shell. Accordingly, CSS-MBs may include an extra polymer shell between the magnetic core and the first shell.

Box 220 may generally be followed by box 230, which describes coating the magnetic core and first shell material with a second shell material. In other words, box 230 may describe coating a second shell material onto the intermediate beads. Box 230 may include decomposing a second shell material in a solution onto the intermediate bead formed during boxes 210 and 220. Box 230 may include mixing the intermediate beads formed as products of box 220 with a second shell material. In some embodiments, the second shell material may be a silicon organic compound (e.g., tetraethyl orthosilicate or tetramethyl orthosilicate) and the decomposition of the second shell material may include hydrolyzation and crosslinking of the silicon organic compounds onto an outer surface of the first shell. For example, the decomposition of the second shell material may be processed in a solvent mixture of water/ethanol at a volume ratio of about 4:1 at room temperature for a period of about 4-24 h. The decomposition of the second shell material onto the intermediate beads may form the CSS-MBs. In some embodiments, after decomposition of the second shell material onto the magnetic beads, the magnetic beads may be washed and dried. For example, the magnetic beads may be washed with a solvent (e.g., ethanol and water) and dried at room temperature, in a process similar to the wash described in box 220. In some embodiments, the magnetic beads may be washed multiple times (e.g., 3-4 times) before being dried.

In some embodiments, box 230 may include forming pores in the second shell (e.g., the outer shell). The process of forming the pores may include mixing the second shell material with a pore template before decomposing the second shell material (and pore template) onto the magnetic core and first shell. The pore template may be a surfactant. For example, a co-assembly of silicon organic compounds (e.g., the second shell material) and surfactant (e.g., the pore template) in a solvent mixture of water/ethanol at a volume ratio of 1:4 may be mixed with the products of box 220 at room temperature for about 4-24 h. After washing the magnetic beads with ethanol and water, forming the pores may include removing the pore template. For example, the pore template of surfactant may be removed by ion exchange to form the pore structure on the second shell.

In some embodiments, the method 200 may also include additional processes such as performing a surface modification on the second shell. In an example surface modification, after box 230, the method 200 may continue with treating the magnetic beads with acid under sonication to maximize hydroxyl groups on the shell. The hydroxyl group on the shell serve as the foundation for further modification. The surface modification may include converting the hydroxyl groups to active sites and binding functional groups to the active sites. For example, the hydroxyl groups are converted to amine groups through the hydrolysis of (3-Aminopropyl) triethoxysilane, followed by covalent attachment of functional groups such as hydrophilic moieties and compounds with epitope of carboxylic groups onto the shell. In some embodiments, the covalent attachment is realized through dehydration synthesis between amine groups on the outer silica shell and carboxylic groups within compounds. In some embodiments, the covalent attachment is realized through click chemistry under the catalysis of copper. The exposed carboxylic groups can selectively bind determined size of biomolecules under certain chemical environment.

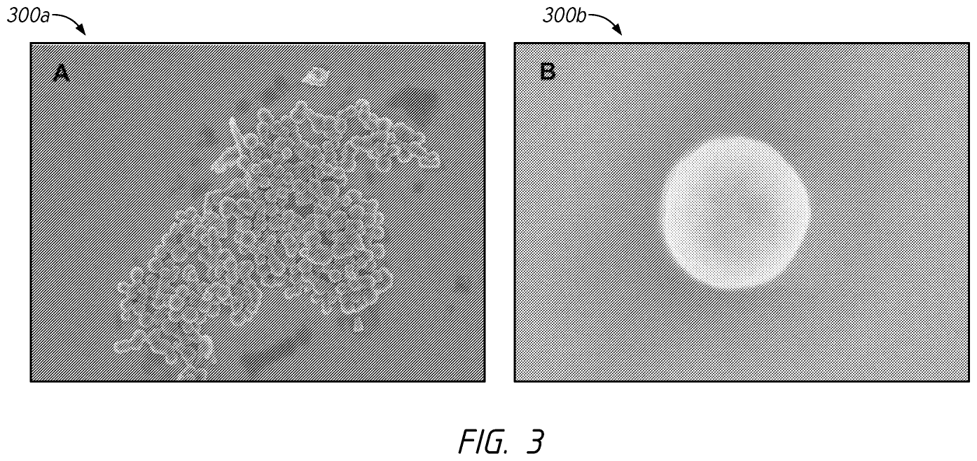

FIGS. 3A to 5 show different examples of data which characterize CSS-MBs (e.g., the CSS-MB 100 of FIG. 1). The FIG. 3A-5 show example data based on a particular synthesis protocol for the CSS-MBs (e.g., some embodiments of the method 200 of FIG. 2). It should be understood that the data shown is for illustrative purposes of a particular example, and that there will be variations both between different batches of the same synthesis protocol and also variations when different synthesis protocols are used.

FIGS. 3A and 3B are electron micrographs of core-shell-shell magnetic beads according to some embodiments of the present disclosure. The micrographs 300a and 300b are both scanning electron micrographs of CSS-MBs (e.g., CSS-MBs 100 of FIG. 1). The micrograph 300a is taken at lower magnification than the micrograph 300b. The micrograph 300a shows a number of CSS-MBs, while the micrograph 300b shows a single CSS-MB.

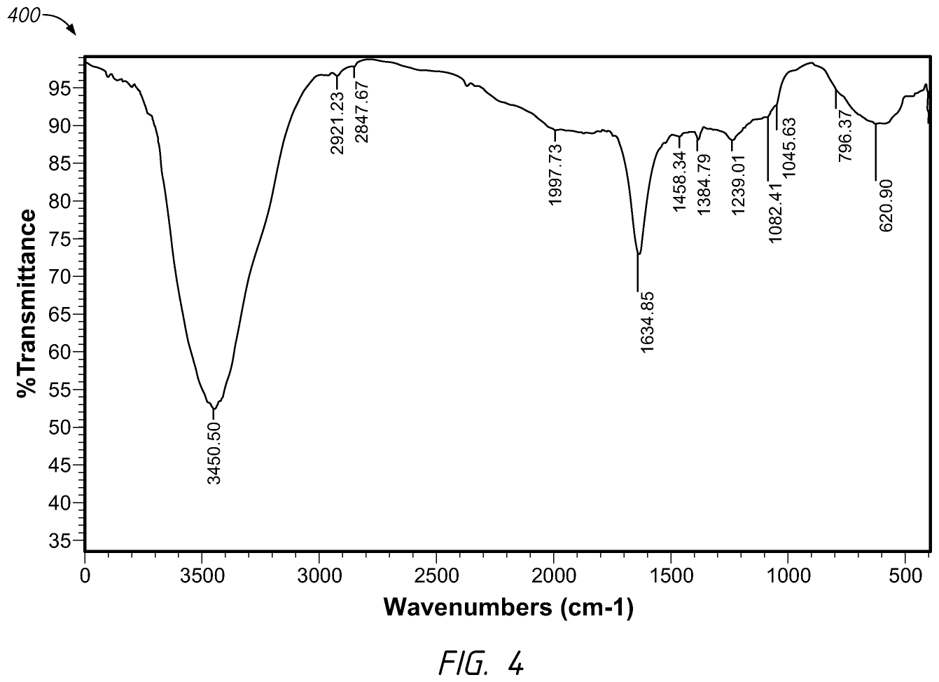

FIG. 4 is a spectrograph of a core-shell-shell magnetic bead according to some embodiments of the present disclosure. The spectrograph 400 shows wavenumber (in cm⁻¹) along the x-axis and % transmittance along the y-axis. The spectrograph 400 is an example of data acquired using a Fourier Transform Infrared Spectrometer. The main peaks which may be seen in the spectrograph 400 show that the primary components of the CSS-MBs are silica, iron, and hydroxy.

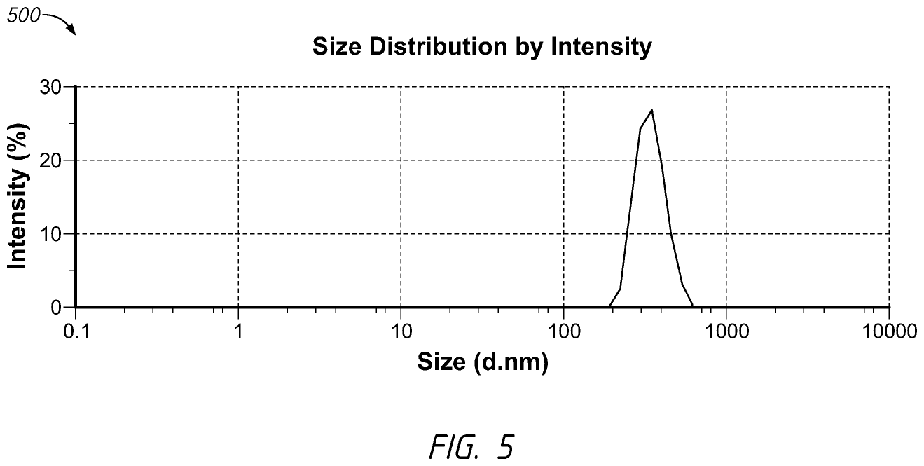

FIG. 5 is a graph of the size distribution of core-shell-shell magnetic beads according to some embodiments of the present disclosure. The graph 500 shows a diameter of the CSS-MB (e.g., CSS-MB 100) in a logarithmic scale along the x-axis and a concentration of the CSS-MBs which are that diameter along the y-axis. As may be seen, the population of CSS-MBs may be highly monodisperse, with the sizes of the CSS-MBs clustered between 200-1000 nm. The relative uniformity of the CSS-MBs, along with their relatively small size (e.g., about 1 μm or less) may increase suitability of the CSS-MBs for suspension in an aqueous environment, which in turn may increase the interaction between the CSS-MBs and biomolecules in the aqueous environment. This may increase the reproducibility of assays conducted using the CSS-MBs.

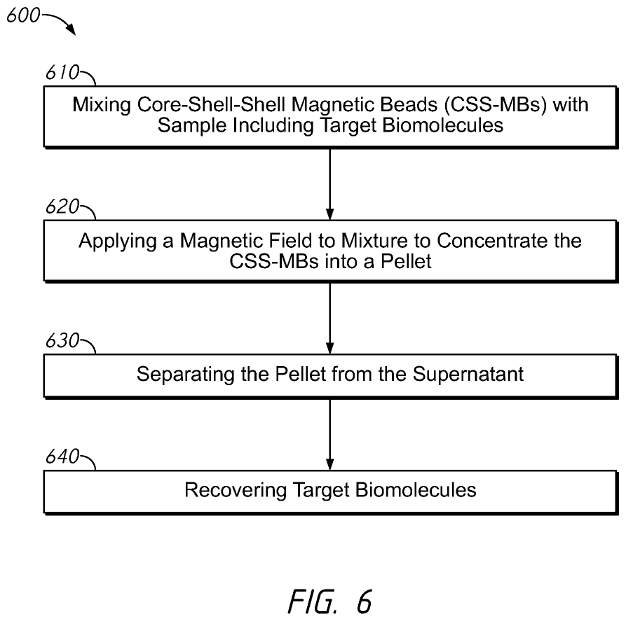

FIG. 6 is a flow chart depicting a method of enriching biomolecules according to some embodiments of the present disclosure. The method 600 may generally be performed using core-shell-shell magnetic beads (e.g., the CSS-MBs 100 of FIG. 1). The method 600 of FIG. 6 is described as a generic process of purifying, enriching, and/or recovering target biomolecules from a sample. Various examples describing example embodiments of the method 600 are provided below.

The method 600 may generally begin with box 610, which describes mixing core-shell-shell magnetic beads with a sample which includes target biomolecules. The term sample may generally refer to any chemical which includes a target biomolecule. The sample may be a mixture or a solution which includes the target biomolecules as well as one or more other chemical components. In general, the sample may include one or more biomolecules in an aqueous solution, however non-aqueous solutions may also be used. In some embodiments, the sample may be a biological sample, such as a fluid obtained from a patient, for example, blood, tissues, serum, plasma, lymph, urine, semen, saliva, milk, cultured cells and combinations thereof. In some embodiments, a biological sample may be obtained from a non-human subject, such as a plant or animal, or may be from an artificial source, such as a cell line cultured in a laboratory. In some embodiments, the sample may be the product of a previous reaction or assay. For example, the sample may be the product of a molecular biology assay such as gene sequencing, an amplification reaction (e.g., qPCR, ddPCR, isothermal amplification, etc.), and/or other processes.

The target biomolecule may be a nucleic acid, proteins, peptides, cells, exosome, or combinations thereof. In some embodiments, the target biomolecule may be a particular type of biomolecule which is distinguished from other biomolecules of the same type. For example, in some embodiments the target biomolecules may be nucleic acids above a certain size cutoff, while nucleic acids below the size cutoff may not be targeted. In some embodiments, the nucleic acids may include DNA, RNA, oligos, labelled nucleic acids such as nucleic acids labeled with radioactive phosphates and/or fluorophores, modified nucleic acids, such as nucleic acids including nucleotides modified with biotin or digoxygenin, or combinations thereof.

The CSS-MBs may be provided as a dry ingredient or may be suspended in a buffer. In some embodiments, the buffer may be a binding buffer to promote binding of one or more molecules to the CSS-MBs. In some embodiments, the CSS-MBs may be combined with the sample, and then a binding buffer may be added separately. In some embodiments, the mixing may involve agitation (e.g., vortexing, stirring, etc.) of the mixture of the sample and the CSS-MBs.

Once the CSS-MBs are combined with the solution, the CSS-MBs may bind to one or more molecules in the solution. For example, in some embodiments, the CSS-MBs may bind to the target biomolecules. In some embodiments, the CSS-MBs may bind to various components of the solution except the target bio-molecule. For example, if the target bio-molecule is DNA below a certain size threshold, then the CSS-MBs may bind to DNA which is above that size threshold.

Block 610 may generally be followed by block 620, which describes applying a magnetic field to the mixture to concentrate the CSS-MBs into a pellet. The applied magnetic field may concentrate the CSS-MBs in a particular region of the container holding the mixture and reduce the concentration of the CSS-MBs in the remainder of the container. For example, the magnetic field may cause the CSS-MBs to precipitate to the particular region of the container which may leave a region with a generally solid pellet of CSS-MBs (and the molecules they are bound to) and a liquid supernatant in the remainder of the container. In some embodiments, the magnetic field may cause substantially all of the CSS-MBs to concentrate into the pellet, and the supernatant may be substantially free of the CSS-MBs.

The magnetic field may be provided by a magnet, such as a solid magnet or an electromagnet. In some examples the magnetic field may be provided by bringing a container holding the mixture in close proximity to a magnet. Responsive to the magnetic field, the CSS-MBs (and the molecules they are bound to) may be drawn into a pellet. For example, in some embodiments where the source of the magnetic field is a magnet held against the container, the CSS-MBs may be drawn to a region of the container closest to the magnet and a pellet may form against the wall of the container closest to the magnet.

Block 620 may generally be followed by block 630, which describes separating the pellet from the supernatant. After the application of the magnetic field, the CSS-MBs (and the molecules they were bound to) may be concentrated into a pellet and the remaining liquid components of the mixture may be a liquid supernatant. The pellet and the supernatant may be separated from each other. For example, the liquid supernatant may be removed from the container (e.g., by pipetting the supernatant out of the container), leaving the solid pellet behind. In some embodiments, the pellet may be removed from the container, and the supernatant may be left behind.

Block 630 may generally be followed by block 640, which describes recovering the target biomolecules. The process of recovering the target biomolecules may depend on the type of target biomolecule and also whether or not the target biomolecules were bound to the CSS-MBs (e.g., in block 610).

In some embodiments where the CSS-MBs bind the target biomolecules, block 640 may involve releasing the target biomolecules from the CSS-MBs. For example, the pellet may be resuspended in a liquid. For example, a liquid may be added to the container which includes the pellet, and then agitated to resuspend the pellet in the liquid. The liquid may, in some embodiments, may be an elution buffer which includes chemistry which causes the target biomolecules to be released from the CSS-MBs into the liquid. Particular chemistry which is useful for releasing the target biomolecules will be discussed in more detail in regards to the specific examples below. In some embodiments, blocks 620 and 630 may then be repeated to remove the (unbound) CSS-MBs, leaving the target biomolecules behind in the liquid.

In some embodiments, the CSS-MBs may bind non-target biomolecules. For example, the CSS-MBs may bind biomolecules in the sample except for the target biomolecules, leaving them unbound in the mixture. In an example embodiment where the target biomolecules are nucleic acids below a certain size threshold, the CSS-MBs may bind to all nucleic acids above that size threshold. Accordingly, after block 630 is performed, the target biomolecules may remain in the supernatant. In some embodiments, the CSS-MBs may bind to substantially all of the non-targeted biomolecules in the sample and the supernatant may include primarily (e.g., only) the targeted biomolecules. In some embodiments where the target biomolecules remain in the supernatant, then block 640 may involve retaining the supernatant.

In some embodiments, before box 610, the method 600 may include preparing the solution. This may involve one or more steps to make the solution more compatible with the method and/or increase the expected yield of the method 600. For example, if the solution is a biological sample containing cells, preparing the solution may include processes such as lysing the cells, removing cellular debris, and/or filtering the cells out of the solution. In another example, if the solution is in a buffer which is not compatible with the CSS-MBs, the method 600 may include performing a buffer exchange on the solution to replace the non-compatible buffer with a compatible buffer. Other types of sample preparation may be used in other example embodiments.

In some embodiments, the method 600 may include recovering the CSS-MBs. It may be desirable to recycle the CSS-MBs so that they can be used in more than one reaction. The recovering of the CSS-MBs may be performed after block 640, and may include releasing any biomolecules (either targeted or non-targeted) which are bound to the CSS-MBs. The recovering may also include washing the CSS-MBs. The recovered CSS-MBs may then be reused as part of a different process of the same assay, or as part of a new assay.

In some embodiments, the method 600 may be used to concentrate a target biomolecule. For example, the method 600 in such an application may include mixing the CSS-MBs with the sample to capture the target biomolecules as part of block 610; separating the CSS-MBs from the solution through an external magnetic field and discarding the supernatant as part of blocks 620 and 630, respectively; washing the magnetic beads; and eluting the target biomolecules from the CSS-MBs in a much smaller elution volume as part of the block 640, to concentrate the target biomolecules. In some embodiments, the concentrated target biomolecules may be resuspended in a buffer different from the buffer they were originally suspended in (e.g., a buffer exchange). In some embodiments, the concentrated target biomolecules may be resuspended in a buffer similar to the one they were originally suspended in.

One example application for the method 600 is the separation and enrichment of biomolecules by size, from the background biomolecules. In such an application, the target biomolecules may be biomolecules above a cutoff size, below a cutoff size, between a range of cutoff sizes, or outside a range of cutoff sizes. For example, the target biomolecule may be nucleic acids above a particular cutoff size (e.g., above about a certain number of base pairs). For example, the size cutoff may be between about 100 bp and 600 bp. Cutoff sizes larger than 600 bp and smaller than 100 bp may also be used in other examples. Different buffer conditions may be used to determine the size of nucleic acid which binds to the CSS-MBs.

In some embodiments, nucleic acids (e.g., DNA, RNA, oligos, etc.) preferentially and reversely bind to the CSS-MBs in the presence of a nucleic acid precipitation buffer, which may include dehydrating agents, salt bridge, buffering agents, and combinations thereof. These nucleic acid precipitation buffer may be part of a buffer that the CSS-MBs are suspended in or may be added to the CSS-MBs. For example, the nucleic acid precipitation buffer may be added to the CSS-MBs and then the sample may be mixed with the suspension of the CSS-MBs as part of block 610. In another example, the CSS-MBs and sample may be mixed, and then the chemicals may be added as part of the block 610.

The dehydrating agents may provide a hydrophobic solution to force hydrophilic nucleic acid molecules out of solution, which may facilitate the precipitation of nucleic acids on the magnetic beads. The salt bridge help minimize the negative charge repulsion of the nucleic acid molecules. The buffering agents provides appropriate pH for nucleic acids to bind on the magnetic beads surface. In some embodiments, the nucleic acid precipitation reagent may include a surfactant, which may reduce non-specific binding of nucleic acids to the CSS-MBs.

In some embodiments, the targeted biomolecule binds to the beads with the presence of dehydrating agents (i.e. polyethylene glycol, polypropylene glycol, etc.), salt bridge (i.e. NaCl, KCl, CaCl₂, etc.), and buffering agent (i.e. Tris, Tris-HCl, EDTA, etc.). In some embodiments, the targeted biomolecule binds to the beads at certain pH (pH from 3 to 8). In some embodiments, the targeted biomolecule is eluted from the beads at low salt concentrations. In some embodiments, the targeted biomolecule is eluted from the beads at certain pH (pH from 6 to 10).

In a non-limiting example, in an application where the target biomolecules are nucleic acids of a certain size, the method 600 may include providing a magnetic bead solution and applying the solution to nucleic acid samples to capture nucleic acid above a cutoff value as part of the block 610. The example application of the method 600 also includes separating the beads from the solution through external magnetic field as part of the block 620 and discarding the supernatant containing the nucleic acids below the cutoff value as part of block 630. The method 600 may include an optional washing of the magnetic beads after the supernatant is discarded and continue with eluting the purified nucleic acid above the cutoff value from the beads as part of the block 640.

In a non-limiting example, in an application where the target biomolecules are nucleic acids below a first cutoff value and above a second cutoff value, the method may include mixing a sample including nucleic acids with the magnetic bead solution to capture nucleic acids above the first cutoff value as part of block 610. For example, buffer conditions in the mixture of the CSS-MBs and the sample may be chosen to cause nucleic acids above the first cutoff value to preferentially bind to the CSS-MBs. The example application of the method 600 may include applying a magnetic field as in block 620, and separating the pellet from the supernatant as in block 630. The example application of the method 600 may include discarding the CSS-MBs which are bound to the nucleic acids above the first cutoff size while collecting the supernatant as part of the block 640. The example application of the method 600 may then repeat back to the block 610 and include applying a new CSS-MB solution to the collected supernatant. The new CSS-MB solution may include buffer conditions which preferentially cause nucleic acids above the second cutoff value to bind to the CSS-MBs. For example, the second buffer conditions may be stronger than the buffer conditions which caused nucleic acids above the first cutoff size to bind to the CSS-MBs. The example application of the method 600 may include separating the beads from the solution through external magnetic field as in blocks 620 and separating the pellet from the supernatant as in block 630; washing the CSS-MBs; and eluting the nucleic acids from the CSS-MBs as in block 640. Accordingly, the nucleic acids which are eluted from the CSS-MBs may be below the first cutoff value and above the second cutoff value.

In some embodiments, one or more of the various reagents which are used in the method 600 may be packaged together in a kit. In some embodiments, the kit may include chemical reagents, such as the CSS-MBs and buffers such as binding buffers and wash buffers. In some embodiments, the kit may include physical components, such as a magnet and various (reusable or disposable) containers for mixing reagents. In some embodiments, the kit may include reagents pre-portioned for a single reaction. In some embodiments, the kit may include reagents which are packaged in larger quantities, and portioning may be up to a user of the kit. In some embodiments, the kit may include instructions which allow the user to ‘tune’ various aspects of the method 600. For example, the kit may include instructions for changing a concentration of a binding buffer, which in turn may change a target size of biomolecule.

An example kit for separating nucleic acids by size may include a population of CSS-MBs (e.g., the CSS-MBs 100 of FIG. 1) and a nucleic acid precipitation buffer. The nucleic acid precipitation buffer may include dehydrating agents, a salt bridge, buffering agents and combinations thereof. The dehydrating agents may provide a hydrophobic solution to force hydrophilic nucleic acid molecules out of solution, which in turn may facilitate the precipitation of long nucleic acids onto the magnetic beads. The dehydrating agents may include polyalkylene glycols (e.g., polyethylene glycol, polypropylene glycol, etc.) which in some embodiments may have a molecular weight of between 1000 to 10,000 Da, and a weight to volume concentration of between 10% to 25%.

The salt bridge may help minimize the negative charge repulsion of the long nucleic acid molecules. The salt bridge may include salts such as NaCl, KCl, CaCl₂, and combinations thereof. In some embodiments, the salt(s) of the salt bridge may have a concentration of between 0.5M to 5M.

The buffering agents may provide appropriate pH for long nucleic acids binding on the magnetic beads surface. The buffering agents may include Tris, Tris-HCl, EDTA, and combinations thereof. One example set of buffering agents included in a nucleic acid precipitation reagent may include Tris with a concentration of 0-10 mM, EDTA with a concentration of 0-1 mM, Tris-HCl with a concentration of 0-10 mM, and combinations thereof.

The nucleic acid precipitation reagent may also include additional components. In some embodiments, a surfactant is added to the nucleic acids precipitation reagent to reduce non-specific nucleic acid binding. For example, surfactants such as Tween-20, Triton X-100, SDS, and combinations thereof may be used. One example set of surfactants may include Tween-20 with a concentration of 0.01/6-0.5%, Triton X-100 with a concentration of 0.01/6-0.5%, SDS with a concentration of 0.1%-1%, and combinations thereof.

In some embodiments, the nucleic acids precipitation reagent may be used to help determine the size cutoff. For example, increasing the strength (reagent concentration, volume to sample volume, etc.) of the nucleic acid precipitation reagent may decrease the cutoff size of the long versus short fragments.

In some embodiments, the kit may also include a wash buffer, which may be used in one or more washing steps. The wash buffer may include an alcohol. In some embodiments, the kit may include an elution buffer. The elution buffer may include a relatively low salt concentration to cause the release of the short DNA fragments. Alternatively and/or additionally, the elution buffer may include a certain pH (e.g., pH from 6 to 10).

### Example 1—Separation of DNA Ladder Standard by Size

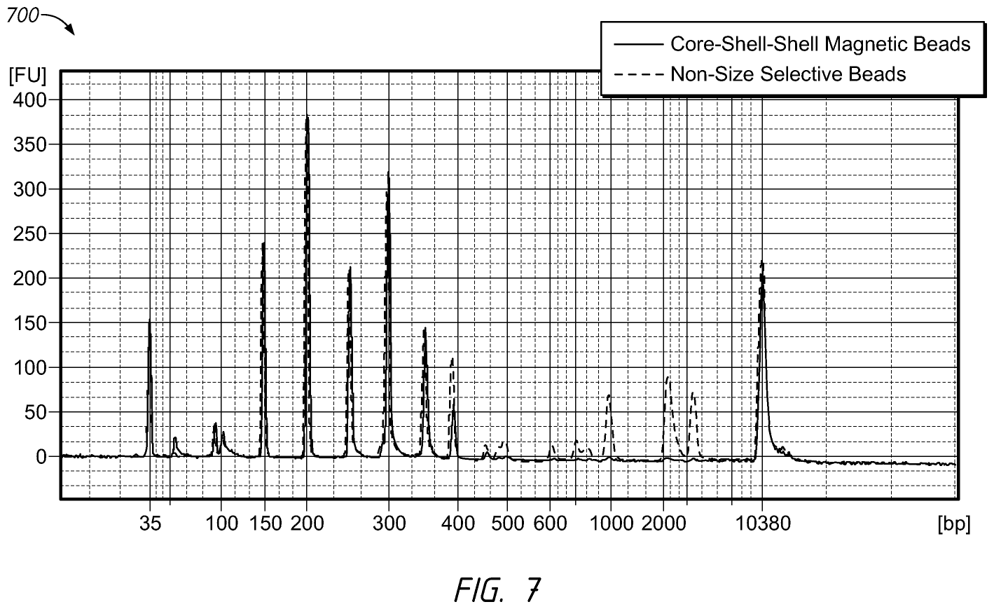

FIG. 7 is a graph of core-shell-shell magnetic beads used to separate nucleic acids by size according to some embodiments of the present disclosure. The graph 700 shows size of nucleic acid along a horizontal axis (in base pairs or bp) and a measure of concentration along the vertical axis (in fractional units, or fu). One of the traces of the graph 700 represents core-shell-shell magnetic beads (CSS-MBs) used to selectively filter out nucleic acids above a cutoff size of 350 bp and below about 100 bp, and the second trace shows the results of non-CSS-MBs used to bind nucleic acids of all sizes in the sample.

The sample of FIG. 7 is a DNA ladder of between about 50-3000 bp. The DNA ladder is treated with CSS-MBs and non-CSS-MBs. The process includes: 1) selectively binding DNA with size >350 bp to the CSS-MBs under a buffer containing dehydrating agent and salt bridge; 2) separating the bound CSS-MBs from the solution under external magnetic field; 3) collecting the supernatant solution containing the unbound DNA with size <350 bp; 4) selectively binding DNA with size of between about 100-350 bp to the magnetic beads under a buffer containing higher concentration of dehydrating agent and salt bridge; 5) separating the CSS-MBs from the solution under external magnetic field and discarding supernatant; 6) washing the magnetic bead with 70% ethanol solution; 7) eluting of the selected DNA from the CSS-MBs with an elution buffer. The enriched DNA is characterized with Agilent 2100 bioanalyzer. The CSS-MBs in the present disclosure removes DNA fragments above the cutoff size (350 bp), and recovers DNA fragments in the range of 100 to 350 bp. The enrichment efficiency is higher than the non-CSS-MBs, with less large fragments' residues but similar recovery of 100 to 350 bp range.

### Example 2—Size Selection on Fragmented DNA

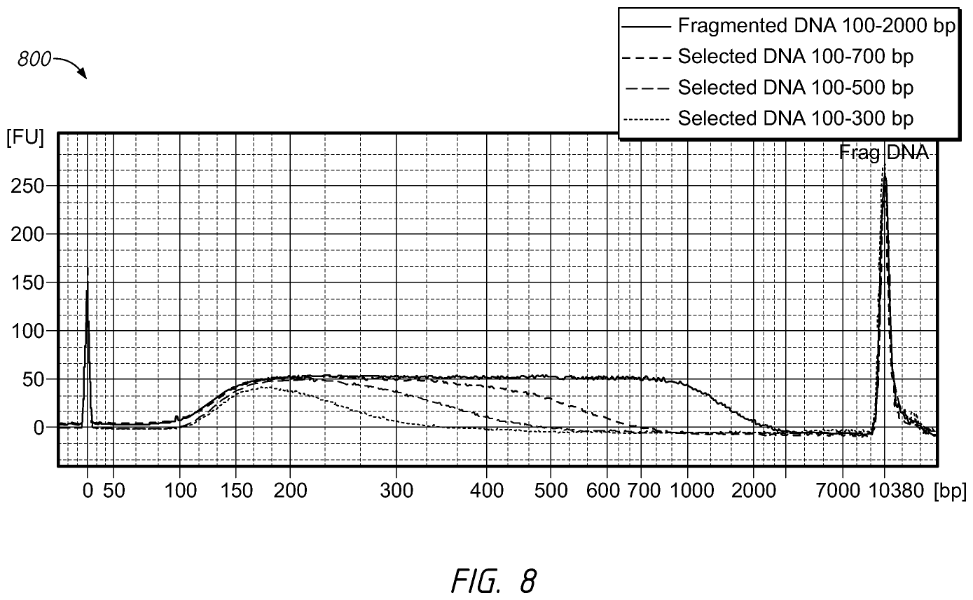

FIG. 8 is a graph of using core-shell-shell magnetic beads to recover nucleic acids between different cutoff ranges according to some embodiments of the present disclosure. The graph 800 is similar to the graph 700, in that it shows the size of nucleic acid along the horizontal axis and concentration at that size along the vertical axis. Each trace of the graph 800 shows a profile of the DNA in a sample when the DNA is filtered using the CSS-MBs for a different size range of DNA. In particular, each experiment uses the same lower cutoff (100 bp) but a different upper cutoff (300, 500, and 700 bp). A trace also shows the unfiltered fragmented DNA which includes DNA fragments between about 100 bp and about 2000 bp.

The example of FIG. 8 is performed using a sample which is derived from cultured human cells. In particular, the sample includes Hela cell's genomic DNA fragmented using a NEBNext® dsDNA Fragmentase® following the manufacture's protocol. The fragmented genomic DNA includes DNA fragments of size in a broad range of about 100 to 2000 bp. The fragmented DNA samples are then treated with CSS-MBs in a manner similar to the method 600 described in FIG. 6. The process includes: 1) selectively binding DNA above an upper cutoff value to the CSS-MBs under a series of buffer conditions containing dehydrating agent and salt bridge of corresponding concentration; 2) separating the CSS-MBs from the solution under an external magnetic field; 3) collecting the supernatant solution containing the unbound DNA; 4) selectively binding DNA with size greater than a lower cutoff (e.g., 100 bp) to the CSS-MBs under a buffer containing a higher concentration of dehydrating agent and salt bridge; 5) separating the CSS-MBs from the solution under an external magnetic field and discarding supernatant; 6) washing the CSS-MBs with 80% ethanol solution; 7) eluting the selected DNA from the CSS-Mbs with an elution buffer. The enriched DNA was characterized with an Agilent 2100 bioanalyzer. The CSS-MBs removes DNA fragments above the cutoff size (300, 500, and 700 bp, respectively), and recovers DNA fragments in the range of 100 bp up to the cutoff size. The CSS-MBs can be used to select DNA fragments with narrow size distribution, suitable for downstream applications.

### Example 3—Clean Up in NGS Library Preparation Workflow

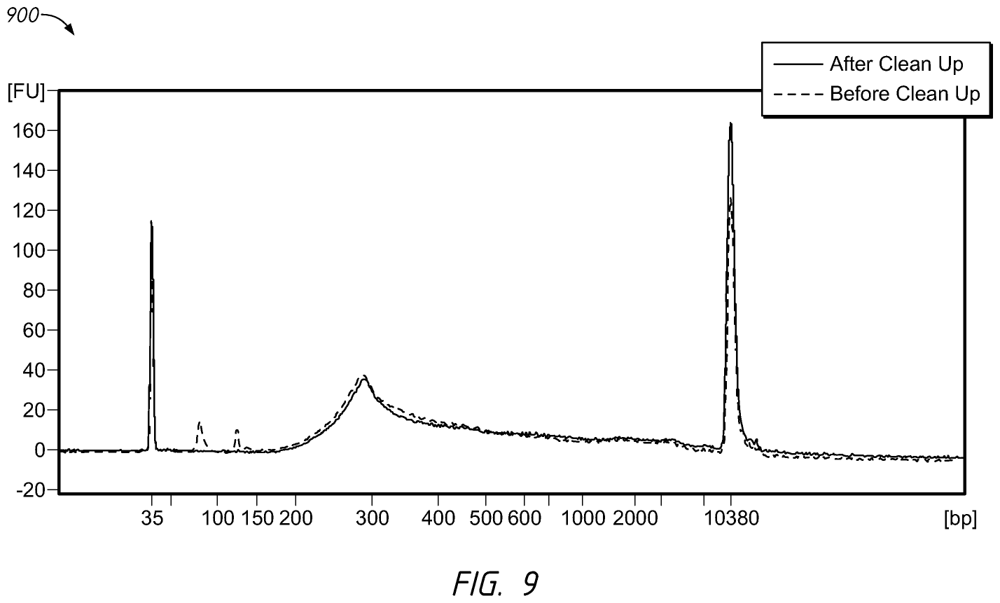

FIG. 9 is a graph of a nucleic acids before and after clean up with core-shell-shell magnetic beads according to some embodiments of the present disclosure. The graph of FIG. 9 represents a clean up procedure on amplified DNA to recover DNA above a cutoff size. The DNA may be amplified using an amplification reaction such as the polymerase chain reaction (PCR). The amplification may be part of a next generation sequencing (NGS) procedure.

A clean up procedure is performed on DNA fragments prepared with adaptor ligation and PCR amplification in a typical NGS library preparation workflow using CSS-MBs (e.g., CSS-MBs 100 of FIG. 1). The library preparation products contain ligated and amplified DNA fragment (>150 bp), and excessive adaptor monomers (50-60 bp) and dimers (120 bp). The process includes: 1) selectively binding DNA with size >120 bp to the CSS-MBs under a buffer containing dehydrating agent and salt bridge; 2) separating the CSS-MBs from the solution under external magnetic field and discard supernatant; 3) washing the CSS-MBs with 70% ethanol solution; 4) eluting the selected DNA from the CSS-MBs with elution buffer. The enriched DNA was characterized with Agilent 2100 bioanalyzer. The beads in the present disclosure removes DNA fragments below the cutoff size of about 120 bp and recovers DNA fragments above about 120 bp.

Accordingly, a CSS-MB may allow for a magnetic bead which has a relatively large magnetic response compared to its size (e.g., due to the solid magnetic core). This in turn, may allow for CSS-MBs to be synthesized at a relatively small size, which may, for example, improve the dispersion of the CSS-MBs in solution as well as increase the surface are to volume ratio. This may improve the performance of the CSS-MBs both in binding to target biomolecules. The use of a first shell material may allow the solid magnetic core to be protected from interaction with components of the solution. This in turn may allow for more freedom in the chemistry of the second shell material, which may be used to further improve the performance of the CSS-MB.

It is to be appreciated that any one of the examples, embodiments or processes described herein may be combined with one or more other examples, embodiments and/or processes or be separated and/or performed amongst separate devices or device portions in accordance with the present systems, devices and methods. For example, any of the methods presented herein may have any of their steps repeated, omitted, or rearranged. The steps of any of the methods may be performed in a different order than the one described. Additional steps may be included in any of the methods presented herein, and may occur between steps which are described herein.

It is to be appreciated that any one of the examples, embodiments or processes described herein may be combined with one or more other examples, embodiments and/or processes or be separated and/or performed amongst separate devices or device portions in accordance with the present systems, devices and methods.

It is to be appreciated that while certain numerical values have been given, these are for example only. A numerical value given as such an example should be considered approximate, and values which are greater or lesser than the example numerical value may also be used. Similarly, when a range of numerical values (e.g., between X and Y) is given, it should be understood to be inclusive of the boundaries (e.g., X and Y) and that boundaries may also be example values which are approximate.

The above-discussion is intended to be merely illustrative of the present system and should not be construed as limiting the appended claims to any particular embodiment or group of embodiments. Thus, while the present system has been described in particular detail with reference to exemplary embodiments, it should also be appreciated that numerous modifications and alternative embodiments may be devised by those having ordinary skill in the art without departing from the broader and intended spirit and scope of the present system as set forth in the claims that follow. Accordingly, the specification and drawings are to be regarded in an illustrative manner and are not intended to limit the scope of the appended claims.

What is claimed is:

1. A magnetic bead comprising:
a solid magnetic core;
a first shell surrounding the solid magnetic core; and
a second shell surrounding the first shell.

2. The magnetic bead of claim 1, wherein the first shell comprises a protective layer.

3. The magnetic bead of claim 1, wherein the first shell is non-porous.

4. The magnetic bead of claim 1, wherein the solid magnetic core comprises a metal alloy oxide with the composition Feₓ—Co_{y}—NiO_{z}.

5. The magnetic bead of claim 1, wherein the first shell comprises an inert carbon shell.

6. The magnetic bead of claim 1, wherein the second shell comprises a silica oxide shell.

7. The magnetic bead of claim 1, wherein the magnetic bead has a diameter of 1 μm or less.

8. The magnetic bead of claim 1, wherein the solid magnetic core has a diameter of 100-300 nm, the first shell has a thickness of 10-50 nm, and the second shell has a thickness of 20-100 nm.

9. The magnetic bead of claim 1, wherein the first shell includes hydroxyl groups, epoxy groups, or combinations thereof.

10. The magnetic bead of claim 1, wherein the second shell includes mesopores.

11. The magnetic bead of claim 10, wherein the mesopores are between 2-20 nm.

12. The magnetic bead of claim 1, further comprising chemical groups disposed on an outer surface of the second shell.

13. The magnetic bead of claim 12, wherein the chemical groups include hydrophilic moieties selected from the group comprising polyethylene glycol, polyacrylic acid, polyvinyl alcohol, glucose, sucrose, cysteine, maltose, chitosan, alginate, cellulose, chitin, starch and combinations thereof.

14. The magnetic beads of claim 12, wherein the chemical groups include compounds with epitope carboxylic groups selected from the group comprising succinic anhydride, polyacrylic acid, amino acids, alginic acid, carbonic acid, malic acid, tartaric acid, citric acid, salicylic acid, gallic acid, sialic acid.

15. A method comprising:
forming a magnetic core;
coating the magnetic core with a first shell material; and
coating the first shell material with a second shell material.

16. The method of claim 15, wherein forming the magnetic core includes heating a mixture of iron salts and salts of at least one other metal under pressure.

17. The method of claim 16, wherein the mixture of iron salts includes Iron (III) Chloride, Iron (III) Sulfate, Iron (III) Nitrate and combinations thereof.

18. The method of claim wherein the mixture of iron salts and salts of at least one other metal includes a surfactant.

19. The method of claim 15, further comprising purifying the magnetic core.

20. The method of claim 15, wherein coating the magnetic core with the first shell material comprises a solvothermal carbonization of a sugar onto the magnetic core.

21. The method of claim 20, wherein the sugar is glucose, sucrose, maltose, or combinations thereof.

22. The method of claim 15, further comprising coating a polymer material onto the magnetic core and wherein coating the magnetic core with the first shell material comprises coating the magnetic core and polymer material with the first shell material.

23. The method of claim 15, wherein coating the first shell material with the second shell material includes decomposing the second shell material in a solution onto an intermediate bead including the magnetic core and first shell material.

24. The method of claim 15, wherein the second shell material comprises a silicon organic compound selected from the group comprising tetraethyl orthosilicate, tetramethyl orthosilicate, and combinations thereof.

25. The method of claim 15, further comprising forming pores in the second shell material.

26. The method of claim 15, further comprising modifying a surface of the second shell material.

27. The method of claim 26, wherein the surface modification includes converting hydroxyl groups on a surface of the second shell material to active sites and binding functional groups to the active sites.

28. A method comprising:
mixing core-shell-shell magnetic beads (CSS-MBs) with a sample including target biomolecules, wherein the CSS-MBs comprise a solid magnetic core, a first shell surrounding the solid magnetic core, and a second shell surrounding the first shell;
applying a magnetic field to the mixture to concentrate the CSS-MBs into a pellet;
separating the pellet from a supernatant of the mixture; and
recovering the target biomolecules from the pellet.

29. The method of claim 28, wherein the target biomolecule includes nucleic acids, proteins, peptides, cells, exosomes, or combinations thereof.

30. The method of claim 28, further comprising binding biomolecules above a cutoff size to the CSS-MBs in the presence of a binding buffer.

31. The method of claim 30, wherein recovering the target biomolecules comprises eluting the target biomolecules from the CSS-MBs in the presence of an elution buffer.

32. The method of claim 30, wherein the target biomolecules are nucleic acids, and wherein the binding buffer includes a nucleic acid precipitation buffer including a dehydrating agent, a salt bridge, a buffering agent, carrier molecules, a surfactant, and combinations thereof.

33. The method of claim 28, further comprising washing the pellet with a wash buffer.

34. A kit comprising:
a population of core-shell-shell magnetic beads (CSS-MB), wherein each CSS-MB includes a solid magnetic core, a first shell material surrounding the solid magnetic core, and a second shell material surrounding the first shell material; and
a nucleic acid precipitation reagent, wherein the CSS-MBs are configured to reversibly bind nucleic acids in the presence of the nucleic acid precipitation reagent.

35. The kit of claim 34, wherein the nucleic acid precipitation reagent includes a dehydrating agent, a salt bridge, a buffering agent, carrier molecules, a surfactant, and combinations thereof.

36. The kit of claim 34, further comprising a wash buffer.

37. The kit of claim 34, further comprising an elution buffer, wherein the CSS-MBs are configured to release the bound nucleic acids in the presence of the elution buffer.
